(19) United States
(12) Patent Application Publication
(10) Pub. No.: US 2026/0204085 A1
(43) Pub. Date: Jul. 16, 2026

(54) TISSUE CELL TREATMENT METHOD AND APPARATUS, ELECTRONIC DEVICE, AND STORAGE MEDIUM

(71) Applicant: STOmics Tech Co., Ltd., Shenzhen (CN)

(72) Inventors: Bohan ZHANG, Shenzhen (CN); Lichuan CHEN, Shenzhen (CN); Mei LI, Shenzhen (CN); Yong ZHANG, Shenzhen (CN); Yuxiang LI, Shenzhen (CN)

(21) Appl. No.: 19/135,295

(22) PCT Filed: Dec. 5, 2022
(86) PCT No.: PCT/CN2022/136568

§ 371 Date: Jun. 3, 2025

(51) Int. Cl. G06V 20/69; G06T 7/00; G06V 10/26; G06V 10/762; G16B 25/10

(52) U.S. Cl. CPC G06V 20/695; G06T 7/0012; G06V 10/267; G06V 10/762; G16B 25/10; G06T 2207/10056; G06T 2207/20021; G06T 2207/30024; G06V 2201/03

(57) ABSTRACT

The present application discloses a tissue cell treatment method and apparatus, an electronic device, and a storage medium. The method comprises: on the basis of a stain image of a target object, determining a labeled region of each tissue type contained in the stain image; according to the cell size of each tissue type, determining the size of a sub-region to be treated corresponding to each labeled region; further performing grid processing on each labeled region to obtain grid coordinates; and extracting corresponding data from gene expression data and overlaying the data on the grid coordinates to obtain a tissue cell treatment result. Since the cell sizes of different tissues are inconsistent, the cell sizes corresponding to different tissue types can be differentiated according to the size of said sub-region, thus generating data of grids of different sizes in different labeled regions to simulate the pseudo cell sizes of different cell tissues, so as to implement tissue-level cell analysis; the obtained analysis result can accurately reflect tissue information of a sample, thus improving the accuracy of the analysis result, and achieving a better analysis effect.

### FIELD

The present disclosure relates to the technical field of transcriptome sequencing, and in particular to a method for processing tissue cells, an apparatus, an electronic device and a storage medium.

### BACKGROUND

A purpose of research in life science is to solve the problem of cells and tissues and how a tissue affects function. Sequencing technology provides gene expression information in some tissues under certain conditions. with the sequencing technology, functions of unknown genes can be inferred, a mechanism of action of certain regulatory genes can be revealed, and cell types and heterogeneity can be identified, providing a theoretical basis for disease diagnosis. Cells, as basic units of a body, cooperate with the micro-environment in specific spatial locations to exert their unique biological functions. Therefore, spatial location information of cells is particularly important for studying and understanding mechanisms in cell biology, tumor biology, developmental biology and other disciplines. Spatial transcriptome may be combined with microscopic imaging and sequencing technology to obtain gene expression data while retaining spatial location information of a sample to the greatest extent. Hence, a spatial transcriptome sequencing technology is proposed.

According to the spatial transcriptome sequencing technology, a gene chip is utilized to retain location information of a sample onto the chip, and then second-generation sequencing technology is applied to sequence the RNA in the sample, and the read content is superimposed back on a tissue image, thereby generating a complete gene expression image of a tissue section. With this technology, spatial location information and gene expression data of cells can be obtained, and thereby research and development of the gene sequencing technology is greatly promoted.

In actual research, data obtained through the spatial transcriptome sequencing technology has errors and cannot meet higher requirements of accuracy.

### SUMMARY

In view of the above, the present disclosure provides a method for processing tissue cells, an apparatus, an electronic device and a storage medium, with which the problem that the conventional spatial transcriptome sequencing technology has errors and results in poor analysis results is solved.

Technical solutions provided for achieving the above objective are described below.

A method for processing tissue cells is provided, comprising:
obtaining gene expression data and a staining image of a target object, wherein the staining image is for providing cell tissue distribution of the target object;
determining, based on the staining image, a marked region and a cell area corresponding to each tissue type of the target object;
determining, based on the cell area, an area of a to-be-processed sub-region in each marked region, wherein the area of the to-be-processed sub-region is positively correlated with the cell area, and the to-be-processed sub-region is for characterizing a minimum processing unit of the corresponding marked region;
gridding the marked region based on the area of each to-be-processed sub-region to obtain a gridded coordinate corresponding to each marked region; and
extracting data corresponding to the gridded coordinate from the gene expression data and superimposing the data on the gridded coordinate to obtain a processing result on tissue cells of the target object.

In a preferred embodiment, the gene expression data comprises a gene identifier, gene coordinate data, and a gene expression level.

In a preferred embodiment, the determining, based on the staining image, a marked region and a cell area corresponding to each tissue type of the target object comprises:
determining the marked region corresponding to each tissue type of the target object based on cell tissue distribution and tissue types in the staining image; and
determining, based on an area and a cell quantity of each marked region, an average cell area as the cell area corresponding to the tissue type; or
obtaining a preset reference cell area of the tissue type as the cell area corresponding to the tissue type.

In a preferred embodiment, the determining, based on the cell area, an area of a to-be-processed sub-region in each marked region comprises:
determining a parameter value corresponding to a to-be-processed sub-region in each marked region based on the cell area and a preset capture unit area, and determining the area of the to-be-processed sub-region based on the parameter value; or
obtaining grid data corresponding to the cell area, wherein the grid data represents dimensions of a square grid with the cell area as a grid area, and determining the area of the to-be-processed sub-region in the corresponding marked region based on the grid data.

In a preferred embodiment, the determining a parameter value corresponding to a to-be-processed sub-region in each marked region based on the cell area and a preset capture unit area, comprises:
determining the number of capture units corresponding to each cell area based on the cell area and the preset capture unit area; and
determining the parameter value of the to-be-processed sub-region in the marked region based on the number of capture units corresponding to each cell area.

In a preferred embodiment, the gridding the marked region based on the area of each to-be-processed sub-region to obtain a gridded coordinate corresponding to each marked region comprises:
dividing each marked region into grids with an area equivalent to that of the to-be-processed sub-regions, and obtaining the center coordinate of each grid, thereby obtaining the gridded coordinate corresponding to the marked region.

In a preferred embodiment, the obtaining the center coordinate of each grid to obtain the gridded coordinate corresponding to the marked region comprises:
obtaining, for each of the grids, a coordinate range where the grid is located, based on coordinates established by capture units; and determining a center point of the coordinate range as a gridded coordinate corresponding to the grid.

In a preferred embodiment, the method further comprises:
performing cell clustering analysis on the target object based on the processing result on tissue cells, and outputting a clustering analysis result.

An apparatus for processing tissue cells is provided, comprising:
a sample pre-processing unit, configured to obtain gene expression data and a staining image of a target object, wherein the staining image is for providing cell tissue distribution of the target object;
a data analysis unit, configured to determine, based on the staining image, a marked region and a cell area corresponding to each tissue type of the target object;
a sub-region determination unit, configured to determine, based on the cell area, an area of a to-be-processed sub-region in each marked region, wherein the area of the to-be-processed sub-region is positively correlated with the cell area, and the to-be-processed sub-region is for characterizing a minimum processing unit of the marked region;
a gridding unit, configured to grid the marked region based on the area of each to-be-processed sub-region to obtain a gridded coordinate corresponding to each marked region; and
a data extraction unit, configured to extract data corresponding to the gridded coordinate from the gene expression data and superimpose the data on the gridded coordinate to obtain a processing result on tissue cells of the target object.

An electronic device is provided, comprising at least one memory and at least one processor, wherein the memory stores an application program, the processor is configured to call the application program stored in the memory, and the application program is for implementing the method for processing tissue cells as described in any one of the above method embodiments.

A storage medium is provided, which stores computer program codes, wherein the computer program codes, when executed, implement the method for processing tissue cells as described in any one of the above method embodiments.

As can be seen from the technical solutions, in the method for processing tissue cells provided in the embodiments of the present disclosure, the gene expression data and the staining image of the target object are obtained, the marked region corresponding to each tissue type in the target object is determined based on the staining image, the size of the to-be-processed sub-region of each marked region is obtained based on the cell size of the tissue type, gridding is further performed on each marked region to obtain a gridded coordinate, and the corresponding data is extracted from the gene expression data, and superimposed on the gridded coordinate, so that the processing result on tissue cells is obtained. In the present disclosure, cell processing and analysis is performed based on cell tissues. Inconsistency of cell sizes in different tissues is considered, sizes of the to-be-processed sub-regions are set to be different to distinguish cell sizes corresponding to different tissue types, thereby grid data of different sizes are generated in different marked regions, and pseudo-cells of different cell tissues are simulated, which achieves tissue-level cell analysis, reflects tissue information of the sample more accurately and achieves a better analysis result.

### BRIEF DESCRIPTION OF THE DRAWINGS

For clearer illustration of the technical solutions according to embodiments of the present disclosure or the prior art, hereinafter briefly described are the drawings to be applied in embodiments of the present disclosure or the prior art. Apparently, the drawings in the following descriptions are only some embodiments of the present disclosure, and other drawings may be obtained by those skilled in the art based on the provided drawings without making any inventive effort.

FIG. 1 is a flow chart of a method for processing tissue cells according to an embodiment of the present disclosure;

FIG. 2 is a schematic diagram of tissue cell expression data according to an embodiment of the present disclosure;

FIG. 3 is a schematic diagram of a tissue cell pattern of an Arabidopsis stem sample according to an embodiment of the present disclosure;

FIG. 4 is a staining image of tissue cells of an Arabidopsis stem sample according to an embodiment of the present disclosure;

FIG. 5 is a schematic diagram of a marking result according to an embodiment of the present disclosure;

FIG. 6a is a schematic diagram of a processing result on tissue cells according to an embodiment of the present disclosure;

FIG. 6b is a schematic diagram of a processing result on tissue cells obtained through a conventional method;

FIG. 7ais a schematic diagram of a clustering result of FIG. 6a;

FIG. 7b is a schematic diagram of a clustering result of FIG. 6b; and

FIG. 8 is a schematic structural diagram of an apparatus for processing tissue cells according to an embodiment of the present disclosure.

### DETAILED DESCRIPTION

Hereinafter technical solutions of embodiments of the present disclosure are described clearly and completely in conjunction with the drawings of the embodiments of the present disclosure. Apparently, the embodiments described below are only some embodiments, rather than all the embodiments of the present disclosure. Any other embodiments obtained by those skilled in the art based on the embodiments in the present disclosure without making any inventive effort shall fall within the protection scope of the present disclosure.

According to a conventional spatial transcription technology, individual cells and molecular information can be spatially positioned and detected. For example, in the Stereo-seq spatial transcriptome sequencing technology, mRNA in a single cell tissue can be captured through a spatiotemporal chip. After two rounds of sequencing, a spatial position of the mRNA sequence and a gene expression level corresponding thereto are respectively determined, thereby achieving spatial positioning and detection of individual cells and molecular information.

It is found from research that in an actual processing procedure, even for the same tested object, different tissue cells contained therein may have different sizes. For example, the hippocampus of a brain and the meristem area of a plant are composed of extremely small cells. For such extremely small cells, the spatial transcription technology has difficulty in accurately imaging cell contours and cannot obtain effective cell contours, so that acquisition of cell spatial information is affected and accuracy of a fusion result of cell gene expression information and spatial position is reduced.

In order to solve this problem, cell sizes of each tissue of a to-be-tested object may be set to be consistent to obtain unified cell analysis data, which reflects an average expression level of cells of each tissue of the sample. However, such processing method causes an analysis result to be unable to accurately reflect biological information of the sample, resulting in large errors and poor analysis results.

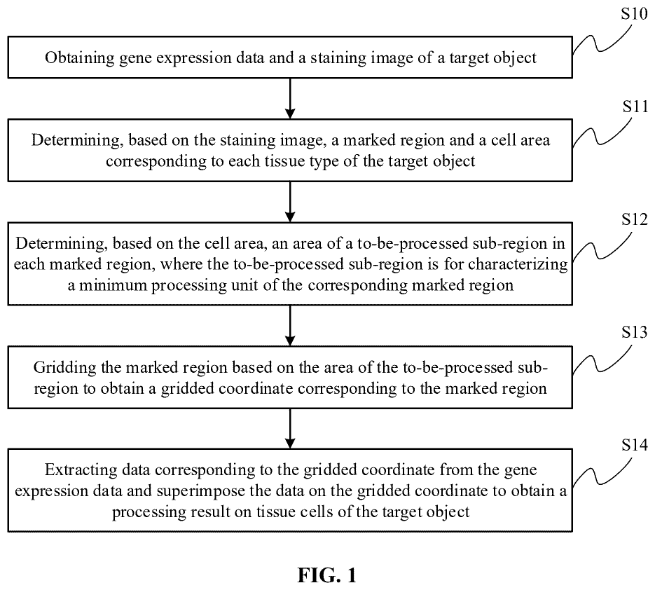

In view of this, a method for processing tissue cells is proposed in the present disclosure. FIG. 1 shows a flow chart of a method for processing tissue cells according to an embodiment of the present disclosure. As shown in FIG. 1, the process may comprise the following steps S10 to S14.

In step S10, gene expression data and a staining image of a target object is obtained.

The target object may be a complete tissue slice of any animal sample or plant sample. The tissue slice may contain multiple cellular tissue structures. For example, a plant sample slice may contain cells of epidermal tissue, phloem tissue, parenchyma tissue and other tissue types.

Particularly, the gene expression data and a registered staining image of the target object may be obtained through the Stereo-seq spatial transcriptome sequencing technology.

The gene expression data of the target object is for recording gene information of the target object, and the registered staining image provides distribution of each cell tissues contained in the target object.

The gene expression data may include a gene identifier ID, gene coordinate data (x, y), and a gene expression level MIDCount.

In step S11, a marked region and a cell area corresponding to each tissue type of the target object are determined based on the staining image.

Particularly, tissue information provided by the staining image may be combined with prior knowledge related to the target object, so that the marked region corresponding to each tissue type included in the target object is determined based on the staining image. The prior knowledge related to the target object may include which cell tissues the target object contains, or the cell morphology of the tissues. In an alternative case, a tissue contour of each cell tissue of the target object may be drawn by a manual tool, and regions corresponding to different tissues may be marked by different colors. The manual tool may be an open-source tool, 3D slicer, which supports manual marking, and may be combined with an open-source tool, plant-seg network, for marking tissue regions.

The cell area in this step may be a reference cell area preset for the tissue type of the target object. That is, each tissue type is set with a cell area in advance. The set cell area may be set based on experience, or may be set based on an average of tissue cells of the same type in different objects. Therefore, in this step, the preset cell area may be directly obtained and used as the cell area corresponding to the marked region, and then the subsequent process is carried out.

In step S12, an area of a to-be-processed sub-region in the marked regions is determined based on the cell area.

The to-be-processed sub-region corresponding to the marked region may serve as a minimum processing unit of the marked region for processing and analysis of the tissue cells. When extracting position information of cells in the target object by using a gene chip, a to-be-processed sub-region is extracted as a single unit, and a positional relationship between to-be-processed sub-regions is used to characterize a positional relationship between cells in the marked region. The area of the to-be-processed sub-region is positively correlated with the cell area, that is, the larger the cell area of the marked region determined in the previous step, the larger the to-be-processed sub-region divided from the marked region. Therefore, a positional relationship among cells in the marked region can be better expressed for different tissue types.

In step S13, the marked region is gridded based on the area of each to-be-processed sub-region to obtain a gridded coordinate corresponding to each marked region.

Particularly, an alternative manner for gridding the marked region is to divide each marked region into grids with an area equal to an area of a to-be-processed sub-region, to obtain gridded coordinates.

Alternatively, the center coordinate of a grid may be determined as a gridded coordinate of the grid. A coordinate range where the grid is located may be obtained from coordinates established by the capture units, and the center coordinate of the coordinate range may be calculated and determined as the center coordinate of the grid.

In step S14, data corresponding to the gridded coordinate is extracted from the gene expression data and superimposed on the gridded coordinate to obtain a processing result on tissue cells of the target object.

Particularly, data corresponding to the gridded coordinate is extracted from the gene expression data to obtain processed data of tissue cells of the target object. The processed data includes expression levels of different genes of the target object in cell tissues of various tissue types. The processed data is further combined with the gridded coordinates to generate the processing result on tissue cells of the target object.

In a possible implementation, the gene expression data includes a gene identifier ID, gene coordinate data (x, y), and a gene expression level MIDCount. As the gridded coordinates are determined, a coordinate range of each grid can be determined through the grid coordinates. Then, each gene coordinate data (x, y) of the data is compared with the coordinate range of each grid. In a case where gene coordinate data (x, y) falls within a coordinate range of a grid, the data is to be superimposed on the grid. In this case, an ID (that is, a cell ID) of the grid corresponding to the data may be set in the expression data. Hence, in the subsequent data superimposing, the grid in which the data falls, that is, a center coordinate of a grid on which the data is to be superimposed, can be determined according to the added cell ID.

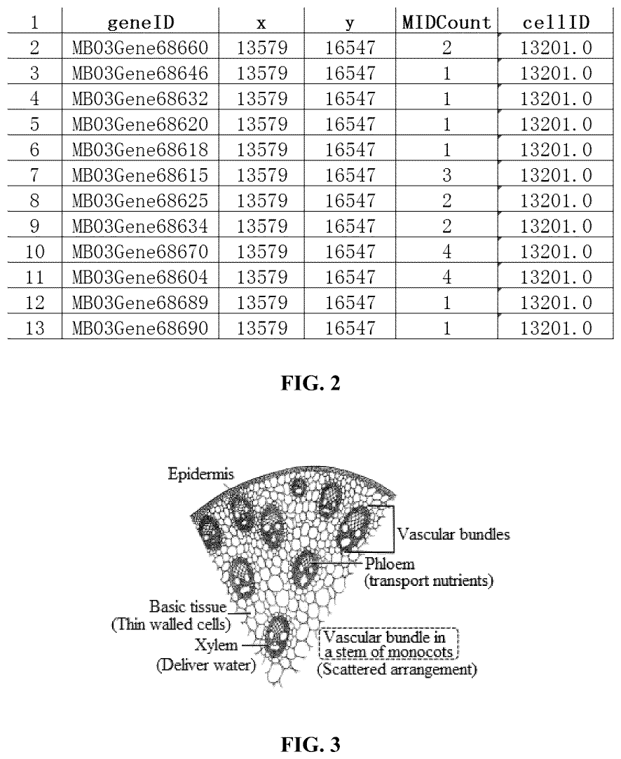

As shown in FIG. 2, gene expression data containing 12 pieces of data are given. Although the data have different gene identifier IDs, cell IDs corresponding thereto are all 13201.0. Therefore, the data is eventually superimposed into a grid whose cell ID is 13201.0.

Alternatively, after the processing result of tissue cells is obtained, a cell clustering analysis may be performed on the target object based on the result, and a result of the clustering analysis may be outputted to visually verify accuracy of the processing result.

In the present disclosure, inconsistency of cell sizes in different tissues is fully considered. Sizes of the to-be-processed sub-regions are determined to reflect cell sizes corresponding to different tissue types, and thereby grid data of different sizes are generated in different marked regions, and cell sizes of different cell tissues are simulated, achieving a tissue-level cell analysis. The obtained analysis result can more accurately reflect tissue information of the sample. As the processing result of tissue cells obtained through the present disclosure is applied to gene sequencing, the accuracy of the analysis result can be improved, thereby achieving a better analysis effect.

Further, the cell areas in step S11 may be obtained through other manners. Particularly, an area of each marked region in the staining image and a cell quantity in each marked region may be determined first, and then an average cell area of each marked region may be calculated and determined as a cell area corresponding to the cell tissue in the marked region.

In this method, information expressed in the staining image is utilized directly as a basis. Thereby, the calculated cell area is more accurate, and the problem of reduced accuracy due to individual differences in target objects is avoided.

In the embodiment, an image processing tool, imagej, may be used for calculating the number of pixels in a marked region, and thereby an area of the marked region is further obtained. In addition, the number of cells in the marked region may be determined, and thereby an average cell area is calculated.

When implementing the step of determining an area of a to-be-processed sub-region in each marked region based on the cell area, considering that the marked region needs to be gridded subsequently, the area of the to-be-processed sub-region may be determined by using grid attributes. Grid data corresponding to the cell area of each marked region may be calculated to obtain the area of the corresponding to-be-processed sub-region. The grid data may include dimensions of a square grid with the cell area. A square grid with an area equal to the cell area may be constructed, and dimensions of the square grid may be used as the grid data. Dimensions and area of the corresponding to-be-processed sub-region may be further determined based on the grid data.

Furthermore, it is considered that a spatial transcriptome sequencing process relies on a gene chip. Therefore, the area of the to-be-processed sub-region in each marked region may be determined based on the cell area in combination with a parameter of the gene chip.

A capture unit of the gene chip is a tool for directly extracting information from a cell tissue. An effect of the information extraction directly affects a final processing effect. Therefore, in an alternative implementation, the area of the to-be-processed sub-region may be determined in combination with an area of the capture unit of the gene chip. First, the cell area corresponding to each marked region is obtained, which is combined with a preset area of the capture unit, so that a parameter value of a to-be-processed sub-region may be obtained. The parameter value may be the number of capture units corresponding to the cell area, that is, indicating how many capture units are needed to capture information in one to-be-processed sub-region, for example, 4900 capture units.

Furthermore, the capture units are arranged in a form of a chip array on the gene chip, and therefore the parameter value may be an arithmetic square root of the number of capture units, that is, indicating the number of the capture units in a form of multiplication, for example, 70*70 capture units.

Next, a method for processing tissue cells according to an embodiment of the present disclosure is described by taking an Arabidopsis stem sample as an example. A tissue cell pattern of the Arabidopsis stem sample is as shown in FIG. 3, which includes pith, phloem, epidermis and other cell tissue structures. As can be seen from FIG. 3, there are obvious differences in cell sizes of different tissue types.

First, gene expression data and a registered staining image of the target object is obtained through the Stereo-seq spatial transcriptome sequencing method.

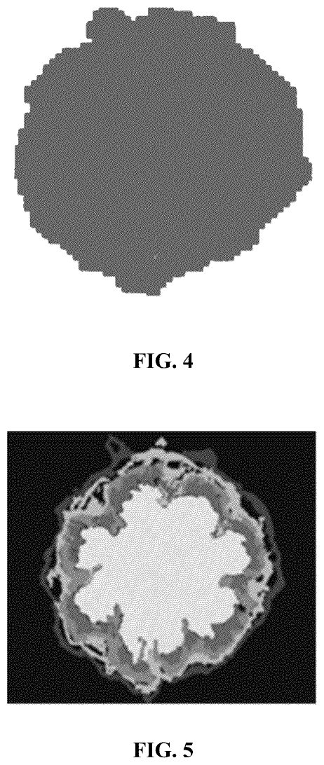

The Stereo-seq sequencing method includes steps of embedding, sectioning, mounting, fixation, permeabilization, reverse transcription, tissue removal, cDNA release, magnetic bead recovery, and the like. In the embodiment, the target object is a cross-section sample slice of Arabidopsis stem. Through the above steps, sample gene expression data and a staining image of the Arabidopsis stem slice can be obtained. The staining image is as shown in FIG. 4.

With the tissue distribution provided in the staining image shown in FIG. 4, based on histological and cellular information such as Arabidopsis stem-related tissue types and cell structures, the biological structure of the Arabidopsis stem sample was obtained, including stele, phloem, epidermis, and other regions of cellular tissue structure. Regions corresponding to each of the above-mentioned cell tissues were marked by different colors or by the same color at different depths through the plant-seg network and the 3Dslicer tool, to determine the marked regions corresponding to each tissue types, respectively. The marking result is as shown in FIG. 5, in which regions of the same color represent the same cell tissue, and the marked regions corresponding to five types of cell tissues were determined. In the case of marking by the same color at different depths, regions marked with the same depth represent the same cell tissue, and different depths indicate different cell tissues.

In the embodiment, the area and the cell quantity of each marked region are determined by using the imagej tool, and an average cell area corresponding to each marked region is calculated.

Then, the average cell area is divided by an area of a capture unit of the gene chip to obtain the number of capture units required for cells in the corresponding marked region. The area of the corresponding to-be-processed sub-region is equal to the total area of captured units occupied by the to-be-processed sub-region. An arithmetic square root of the number of capture units is further calculated and determined as a parameter value of the to-be-processed sub-region.

In the embodiment, parameter values of the to-be-processed sub-regions in the 5regions are 70, 28, 33, 44, and 38, respectively. Taking the marked region with a parameter value of 70 as an example, a to-be-processed region in the region requires 70*70, that is, 4900 capture units.

Next, based on the area of the to-be-processed sub-region corresponding to each marked region, grids are divided in each marked region, and gridded coordinate data is obtained by calculating center coordinates corresponding to the grids, respectively.

In this step, the parameter value of the to-be-processed sub-region corresponding to each marked region are inputted into a preset gridding program, and processed by the gridding program to obtain the gridded coordinate data.

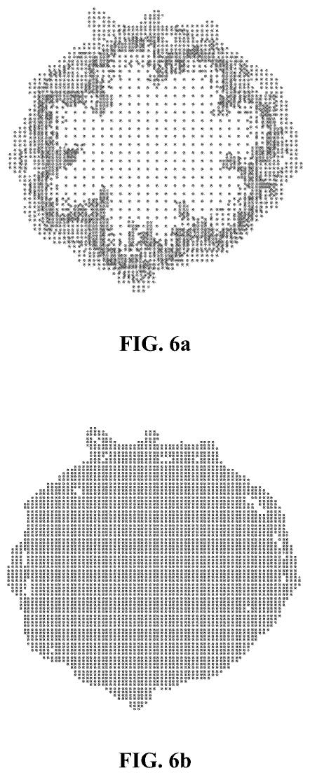

Next, data corresponding to the gridded coordinate are extracted from the gene expression data of the Arabidopsis stem slice sample and superimposed on the grid coordinates to obtain a processing result on tissue cells of the Arabidopsis stem sample. The result is as shown in FIG. 6a.

In FIG. 6a, data points corresponding to the marked region containing small-sized cells in FIG. 2 are denser, and data points corresponding to the marked region containing large-sized cells are sparser. That is, the amount of data extracted from the gene expression data is directly related to the density and sparseness of the grids. The denser the grids, the more the center coordinates, and the more the data extracted. Therefore, the biological information of a tissue with dense cells is reflected.

A gridded result through a conventional processing method is as shown in FIG. 6b, in which grids, regardless of the regions, all have the same size. In this case, there may be omissions in biological information of a tissue with dense cells, which affects a final result.

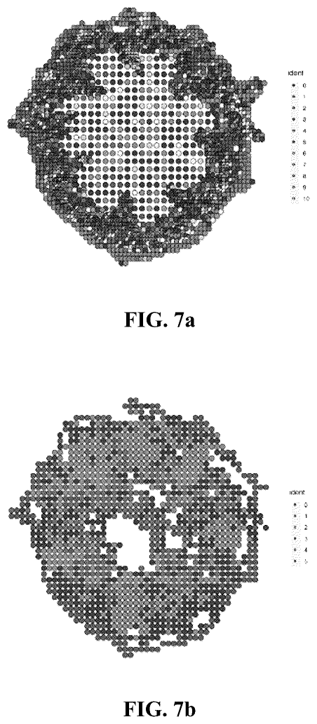

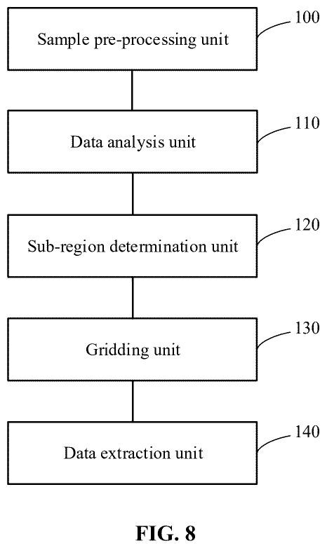

In order to better illustrate the effect of the method for processing tissue cells provided in the present disclosure, clustering is performed on FIG. 6a and FIG. 6b, and FIG. 7a and FIG. 7b are obtained.

From a comparison between FIG. 7a and FIG. 7b, a clustering result obtained after clustering the processing result obtained through the method for processing tissue cells provided in the embodiment of the present disclosure more accurately reflects tissue information of the Arabidopsis stem sample and reconstitutes the biological information of the sample and spatial position information of the tissue cells.

As can be seen from the above, in the present disclosure, cell processing and analysis is performed based on cell tissues. Since inconsistency of cell sizes in different tissues is considered, sizes of the to-be-processed sub-regions are set to be different to distinguish cell sizes corresponding to different tissue types, and thereby grid data of different sizes are generated in different marked regions, and pseudo-cells of different cell tissues are simulated, which achieves tissue-level cell analysis, more accurately reflects tissue information of the sample, and achieves a better analysis result.

Hereinafter described is an apparatus for processing tissue cells according to an embodiment of the present disclosure. Cross reference may be made between the apparatus described below and the method described above.

An apparatus for processing tissue cells is described in conjunction with FIG. 8. As shown in FIG. 8, the apparatus may include a sample pre-processing unit 100, a data analysis unit 110, a sub-region determination unit 120, a gridding unit 130, and a data extraction unit 140.

The sample pre-processing unit 100 is configured to obtain gene expression data and a staining image of a target object, where the staining image is for providing cell tissue distribution of the target object.

The data analysis unit 110 is configured to determine, based on the staining image, the marked region and the cell area corresponding to each tissue type of the target object.

The sub-region determination unit 120 is configured to determine, based on the cell area, an area of a to-be-processed sub-region in each marked region, where the area of a to-be-processed sub-region is positively correlated with the cell area, and the to-be-processed sub-region is for characterizing a minimum processing unit of the marked region.

The gridding unit 130 is configured to grid the corresponding marked region based on the area of each to-be-processed sub-region to obtain a gridded coordinate corresponding to each marked region.

The data extraction unit 140 is configured to extract data corresponding to the gridded coordinate from the gene expression data and superimpose the data on the gridded coordinate to obtain a processing result on tissue cells of the target object.

Alternatively, the data analysis unit 110 may include a tissue region division unit, a cell area determination unit, and a cell area obtaining unit.

The tissue region division unit is configured to determine the marked region corresponding to each tissue type of the target object based on cell tissue distribution and tissue types in the staining image.

The cell area determination unit is configured to determine, based on an area and a cell quantity of each marked region, an average cell area as the cell area corresponding to the tissue type.

The cell area obtaining unit is configured to obtain a preset reference cell area of each tissue type as the cell area corresponding to the tissue type.

Alternatively, the sub-region determination unit 120 may include a parameter value determination unit and a sub-region dimension determination unit.

The parameter value determination unit is configured to determine a parameter value corresponding to a to-be-processed sub-region in each marked region based on the cell area and a preset capture unit area, and determine the area of the to-be-processed sub-region based on the parameter value.

The sub-region dimension determination unit is configured to obtain grid data corresponding to each cell area, where the grid data represents dimensions of a square grid with the cell area as a grid area, and determine the area of a to-be-processed sub-region in the corresponding marked region based on the grid data.

Alternatively, the parameter value determination unit may include a capture unit quantity determination unit and a parameter value calculation unit.

The capture unit quantity determination unit is configured to determine the number of capture units corresponding to each cell area based on the cell area and the preset capture unit area.

The parameter value calculation unit is configured to determine the parameter value of a to-be-processed sub-region in the corresponding marked region based on the number of capture units corresponding to the cell area.

Alternatively, the gridding unit 130 may include a gridded coordinate determination unit.

The gridded coordinate determination unit is configured to divide each marked region into grids each with an area corresponding to an area of a to-be-processed sub-region, and obtain the center coordinates of each grid to obtain the gridded coordinate corresponding to the marked region.

Alternatively, the gridded coordinate determination unit may include a center coordinate calculation unit.

The center coordinate calculation unit is configured to obtain, for each of the grids, a coordinate range where the grid is located, based on the coordinates established by capture units; and determine a center point of the coordinate range as a gridded coordinate corresponding to the grid.

Alternatively, the apparatus for processing tissue cells according to the present disclosure may further include a clustering analysis unit.

The clustering analysis unit is configured to perform cell clustering analysis on the target object based on the processing result on tissue cells, and output a clustering analysis result.

Work flows of the above modules and units may refer to the aforementioned method embodiment and are not repeated here.

With the apparatus for processing tissue cells according to the embodiment, cell processing and analysis is performed based on cell tissues. Since inconsistency of cell sizes in different tissues is considered, sizes of the to-be-processed sub-regions are set to be different to distinguish cell sizes corresponding to different tissue types, thereby grid data of different sizes are generated in different marked regions, and pseudo-cells of different cell tissues are simulated, which achieves tissue-level cell analysis, more accurately reflects tissue information of the sample, and achieves a better analysis result.

An electronic device is further provided in the present disclosure. The electronic device includes at least one memory and at least one processor. The memory stores an application program, and the processor is configured to call the application program stored in the memory to implement the method for processing tissue cells as described in the above method embodiment.

A storage medium is further provided in the present disclosure. The storage medium stores computer program codes. The computer program codes, when executed, implement the method for processing tissue cells as described in the above method embodiment.

It should be further noted that in the present disclosure, the relationship terminologies such as “first” and “second” are only used to distinguish one entity or operation from another entity or operation, rather than to necessitate or imply an actual relationship or order between the entities or operations. In addition, terms “include”, “comprise” or any variants thereof are intended to be non-exclusive. Therefore, a process, method, article or device including a series of elements includes not only the elements but also other elements that are not enumerated, or further includes elements inherent to the process, method, article or device. Unless expressly limited otherwise, the phrase “comprising (including) a/an . . . ” does not exclude existence of other similar elements in the process, method, article or device.

The embodiments in the present disclosure are described in a progressive manner, and each of the embodiments focuses on its differences from the other embodiments. The same or similar parts among the embodiments may be referred to each other.

The above description of the disclosed embodiments enables those skilled in the art to implement or use the present disclosure. Various modifications to the embodiments are apparent to those skilled in the art, and the generic principles defined herein may be practiced in other embodiments without departing from the spirit or scope of the present disclosure. Therefore, the present disclosure is not to be limited to the embodiments shown herein but is to be conformed with the widest scope consistent with the principles and novel features disclosed herein.

1. A method for processing tissue cells, comprising:
obtaining gene expression data and a staining image of a target object, wherein the staining image is for providing cell tissue distribution of the target object;
determining, based on the staining image, a marked region and a cell area corresponding to each tissue type of the target object;
determining, based on the cell area, an area of a to-be-processed sub-region in each marked region, wherein the area of the to-be-processed sub-region is positively correlated with the cell area, and the to-be-processed sub-region is for characterizing a minimum processing unit of the corresponding marked region;
gridding the marked region based on the area of each to-be-processed sub-region to obtain a gridded coordinate corresponding to each marked region; and
extracting data corresponding to the gridded coordinate from the gene expression data and superimposing the data on the gridded coordinate to obtain a processing result on tissue cells of the target object.

2. The method according to claim 1, wherein the gene expression data comprises a gene identifier, gene coordinate data, and a gene expression level.

3. The method according to claim 2, wherein the determining, based on the staining image, a marked region and a cell area corresponding to each tissue type of the target object comprises:
determining the marked region corresponding to each tissue type of the target object based on cell tissue distribution and tissue types in the staining image; and
determining, based on an area and a cell quantity of each marked region, an average cell area as the cell area corresponding to the tissue type; or
obtaining a preset reference cell area of the tissue type as the cell area corresponding to the tissue type.

4. The method according to claim 3, wherein the determining, based on the cell area, an area of a to-be-processed sub-region in each marked region comprises:
determining a parameter value corresponding to a to-be-processed sub-region in each marked region based on the cell area and a preset capture unit area, and determining the area of the to-be-processed sub-region based on the parameter value; or
obtaining grid data corresponding to the cell area, wherein the grid data represents dimensions of a square grid with the cell area as a grid area, and determining the area of the to-be-processed sub-region in the corresponding marked region based on the grid data.

5. The method according to claim 4, wherein the determining a parameter value corresponding to a to-be-processed sub-region in each marked region based on the cell area and a preset capture unit area, comprises:
determining the number of capture units corresponding to each cell area based on the cell area and the preset capture unit area; and
determining the parameter value of the to-be-processed sub-region in the marked region based on the number of capture units corresponding to each cell area.

6. The method according to claim 2, wherein the gridding the marked region based on the area of each to-be-processed sub-region to obtain a gridded coordinate corresponding to each marked region comprises:
dividing each marked region into grids with an area equivalent to that of the to-be-processed sub-regions, and obtaining the center coordinate of each grid, thereby obtaining the gridded coordinate corresponding to the marked region.

7. The method according to claim 6, wherein the obtaining the center coordinate of each grid to obtain the gridded coordinate corresponding to the marked region comprises:
obtaining, for each of the grids, a coordinate range where the grid is located, based on coordinates established by capture units; and determining a center point of the coordinate range as a gridded coordinate corresponding to the grid.

8. The method according to claim 2, further comprising:
performing cell clustering analysis on the target object based on the processing result on tissue cells, and outputting a clustering analysis result.

9. An apparatus for processing tissue cells, comprising:
a sample pre-processing unit, configured to obtain gene expression data and a staining image of a target object, wherein the staining image is for providing cell tissue distribution of the target object;
a data analysis unit, configured to determine, based on the staining image, a marked region and a cell area corresponding to each tissue type of the target object;
a sub-region determination unit, configured to determine, based on the cell area, an area of a to-be-processed sub-region in each marked region, wherein the area of the to-be-processed sub-region is positively correlated with the cell area, and the to-be-processed sub-region is for characterizing a minimum processing unit of the marked region;
a gridding unit, configured to grid the marked region based on the area of each to-be-processed sub-region to obtain a gridded coordinate corresponding to each marked region; and
a data extraction unit, configured to extract data corresponding to the gridded coordinate from the gene expression data and superimpose the data on the gridded coordinate to obtain a processing result on tissue cells of the target object.

10. An electronic device, comprising at least one memory and at least one processor, wherein the memory stores an application program, the processor is configured to call the application program stored in the memory, and the application program is for implementing the method for processing tissue cells according to claim 1.

11. A storage medium, storing computer program codes, wherein the computer program codes, when executed, implement the method for processing tissue cells according to claim 1.
